(19) United States
(12) Patent Application Publication
(10) Pub. No.: US 2023/0372583 A1
(43) Pub. Date: Nov. 23, 2023

(54) COMPOSITIONS AND METHODS FOR TREATING FACIAL VOLUME DEFECTS

(71) Applicant: Francesco BERNARDINI, Genova (IT)

(72) Inventor: Francesco BERNARDINI, Genova (IT)

(21) Appl. No.: 17/749,876

(22) Filed: May 20, 2022

(51) Int. Cl. A61L 27/20

(52) U.S. Cl. CPC A61L 27/20; A61L 2400/06; A61L 2430/34

(57) ABSTRACT

In alternative embodiments, provided are methods for modifying a facial profile, or for treating facial volume defects, comprising administration of cross-linked polysaccharide polymers, or polysaccharide polymer salts, such as hyaluronic acid-based formulations. In alternative embodiments, provided are methods for treating or ameliorating facial volume defects and unwanted static or dynamic lines (lines that are caused by facial expressions such as smiling, laughing, frowning or squinting), including crow's feet, forehead line or any unwanted facial line. In alternative embodiments, methods as provided herein administer cross-linked fillers that are injected superficially at predetermined facial injection sites, such as into the mid-facial region, without reaching the deep fat layer. In alternative embodiments, methods as provided herein treat or ameliorate or modify unattractive nasolabial folds. In alternative embodiments, the cross-linked polysaccharide polymers used to practice methods as provided herein are “resilient”, or have a crosslinker rate (Degree of Modification (MoD)), or rate of crosslinking, of between about 0.1% and 5%, 0.2% and 4.5%, 0.4% and 3%, 0.8% and 2%, 1% and 1.5%, or about 1.5% and 4%, or between about 2% and 3%.

### TECHNICAL FIELD

This invention generally relates to tissue engineering. In alternative embodiments, provided are methods for modifying a facial profile, or for treating facial volume defects, comprising administration of cross-linked polysaccharide polymers, or polysaccharide polymer salts, such as hyaluronic acid-based formulations. In alternative embodiments, provided are methods for treating or ameliorating facial volume defects and unwanted static or dynamic lines (lines that are caused by facial expressions such as smiling, laughing, frowning or squinting), including crow's feet, forehead line or any unwanted facial line. In alternative embodiments, methods as provided herein administer cross-linked fillers that are injected superficially at predetermined facial injection sites, such as into the mid-facial region, without reaching the deep fat layer. In alternative embodiments, methods as provided herein treat or ameliorate or modify unattractive nasolabial folds. In alternative embodiments, the cross-linked polysaccharide polymers used to practice methods as provided herein are “resilient”, or have a crosslinker rate (Degree of Modification (MoD)), or rate of crosslinking, of between about 0.1% and 4%, 0.4% and 3%, 0.8% and 2%, 1% and 1.5%, or about 1.5% and 4%, or between about 2% and 3%.

### BACKGROUND

Currently used techniques for injecting fillers, particularly hyaluronic acid-based fillers, into the face include use of a deep fat (DF) compartment injection technique which involves release of the filler under a fibrous and muscular plane represented by the SMAS layer (Superficial Muscolar Aponerotic System). The SMAS is a fibrous and muscular layer located high in the cheek and in front of the ear, and extends downward enveloping the muscles of facial expression, facial fat compartments, and continues to the jaw and neck. The SMAS layer lies beneath a thin layer of fat and skin, and its purpose is to help the muscles of facial expression move and stay in the correct position. Below the SMAS, a layer of deep fat (DF).

Recent studies have shown that the first signs of facial aging due to fat atrophy occur mainly at the level of the superficial fat compartment. These studies show that in people aged between the second and third decade (between 28 and 40 years old) the atrophy of deep fat has not yet begun.

### SUMMARY

In alternative embodiments, provided are kits and methods for facial tissue engineering comprising administering (or components for administering, for the kits) to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face. In alternative embodiments, methods and kits as provided herein are used for treating or ameliorating facial volume defects and unwanted static or dynamic lines (lines that are caused by facial expressions such as smiling, laughing, frowning or squinting), including crow's feet, forehead line or any unwanted facial line.

In alternative embodiments, provided are methods for tissue engineering comprising administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin. In alternative embodiments, methods and kits as provided herein are used for treating or ameliorating volume defects in the skin, including skin on the arms, legs or torso, including defects due to injuries or burns.

In alternative embodiments of methods as provided herein:
the crosslinked polysaccharide comprises a crosslinked hyaluronic acid polysaccharide, and optionally the crosslinked polysaccharide comprises a salt of a polysaccharide, and optionally the crosslinked polysaccharide salt comprises a sodium salt, a potassium salt, a zinc salt and/or a silver salt or a mixture thereof, and optionally the crosslinked polysaccharide has a crosslinker rate, or Degree of Modification (MoD), or rate of crosslinking, of between about 0.1% and 10%, 0.4% and 8%, 0.8% and 6%, 1% and 5%, or about 1.5% and 4%, or between about 2% and 4%, or the filler has a crosslinker rate of about 0.1%, 0.2%, 0.3%, 0.4%, 0.5%, 0.6%, 0.7%, 0.8%, 0.9%, 1%, 1.5%, 2%, 2.5%, 3%, 3.5%, 4% or 4.5%;
the polysaccharide is crosslinked with or using an epoxide, an aldehydes, a polyaziridyl compound, divinyl sulphone (DVS) and/or butanediol diglycidyl ether (BDDE), or a mixture thereof, and optionally the polysaccharide is crosslinked with or using: 1,4-butanediol diglycidyl ether (BDDE), diepoxyoctane or 1,2-bis(2,3-epoxypropyl)-2,3 -ethylene, 1,4-bis (-epoxypropoxy) butane, 1,4-bisglycidyloxybutane, 1,2-bis (2,3-epoxypropoxy) ethylene, and/or 1-(2,3-epoxypropyl)-2,3-epoxycyclohexane, or a mixture thereof;
the method comprises administering or injecting subcutaneously into the superficial fat of the middle cheek compartment and/or medial cheek compartment (as illustrated in FIG. 5) an amount of filler formulation sufficient to cause stretching and supporting of the mid-facial tissues, thus allowing indirect treatment of the nasolabial fold (NLF), and bringing a volumizing effect to the cheek, and/or optionally, the method comprising:
   (a) administering or injecting subcutaneously at least one bolus (optionally from about 0.05 mL (0.05 cc) to about 0.3 mL (0.3 cc) filler formulation) in or at a lateral facial area, and/or into a middle cheek fat compartment near, or at a site between about 0.5 to 5 mm, or within about 1, 2, 3, 4 or 5 mm of, the zygomatic cutaneous ligament (ZCL), optionally as shown in FIG. 5; and/or
   (b) administering or injecting subcutaneously at least one bolus (optionally from about 0.05 mL (0.05 cc) to about 0.3 mL (0.3 cc) filler formulation) into the upper zone of the medial cheek fat compartment in the area of its major atrophy or depression;
the method comprises administering or injecting subcutaneously into the superficial fat in an upper zone of a middle cheek fat compartment at a site between about 0.5 to 5 mm, or within about 1, 2, 3, 4 or 5 mm, of a zygomatic cutaneous ligament (ZCL) a first bolus of the filler formulation comprising a volume from 0.05 mL (0.05 cc) filler to 0.3 mL (0.3 cc) of the filler formulation;
the method further comprises injecting filler formulation subcutaneously into the superficial fat one, two or three or more additional boluses (or injecting a second, third or fourth or additional bolus or boluses) in the upper zone of the middle cheek fat compartment, wherein optionally the one, two, three, four or more boluses of filler formulation are administered or injected in a volume of between about 0.05 ml to 1.5 ml., or between about 0.10 ml to 1 ml., or between about 0.025 ml to 2 ml.;
the second and/or third bolus or additional boluses of filler formulation is/are administered or injected subcutaneously into the superficial fat at sites as illustrated in FIG. 7 and FIG. 8 and/or approximately equidistant from previous administrations or injection of filler formulation;
the first bolus of filler formulation is administered or injected at approximately at a Gpoint as derived by plotting:
a first connecting line (G1) between the lower end of the nasal wing and the tragus,
a second connecting line (G2) between the lateral end of the eye and the corner of the mouth,
a third connecting line (G3) that crosses the second connecting line G2, and
a fourth connecting line (G4) drawn from the intersection of the first G1 and the second G2 connecting line until it intersects the third G3 connecting line at a 90° angle;
a second bolus of filler formulation is administered or injected into a middle cheek compartment, anteriorly and lower than the Gpoint, following the zygomatic cutaneous ligament (ZCL), as shown in FIG. 8, and/or a third or fourth or further bolus of filler formulation is administered or injected into the middle cheek compartment, posterior to the Gpoint, following the zygomatic cutaneous ligament (ZCL), as shown in FIG. 8;
the method further comprises injecting filler formulation subcutaneously into the superficial fat one, two or three or more additional boluses (or injecting a second, third or fourth or additional bolus or boluses) in the upper zone of the medial cheek fat compartment, wherein optionally the one, two, three, four, five or six or more boluses of filler formulation are administered or injected in a volume of between about 0.05 ml to 1.5 ml., or between 0.025 ml to 2 ml.;
the method further comprises administering (or injecting) in the upper part of the cheek, optionally between the inferior orbital compartment and the medial or middle cheek compartment, a filler formulation following at least one retrotracing line arranged as a bridge between the two superficial fat compartments, and/or optionally the retrotracing lines are practiced in about three (3) to ten (10) retrotracing line sites, or between about 6 to 8 retrotracing line sites, wherein optionally the filler comprises RHA1™ or equivalent, RHA2™ or equivalent, RHA3™ or equivalent or RHA4™ or equivalent;
the method comprises administering (or injecting) filler formulation to enhance a plumping effect of a lower eye contour, wherein the filler formulation is injected according to retrotracing lines arranged between two adjacent boluses;
the method further comprises administering (or injecting) filler formulation into a deep or mid dermis at a nasolabial fold, and optionally the filler formulation is infiltrated (administered, injected) with at least one retrotracing line, and optionally the administering (or infiltrating, or injecting) filler formulation is performed using a bridging technique of the defect, and optionally a needle, or cannula, enters laterally into the cheek side of a defect, passes through it, and into the lip side, and release of the filler formulation is initiated in a linear retreating fashion, also called retrotracing mode, and the nasolabial fold is thus crossed transversely by the filler, and the release is finished as soon as it reaches the cheek side;
the method further comprises, for example, to improve a volumizing effect, administering (or infiltrating, or injecting) one or more boluses filler formulation at a deep or mid intradermal level or deep/mid-dermis, optionally administering (or infiltrating, or injecting) one, two, three or more boluses, optionally 0.05 mL in volume size, and optionally the filler formulation comprises an RHA3™ or equivalent filler injected into a deep or mid dermis plane;
the method further comprises, for example, to fill in a wrinkle, administering (or infiltrating, or injecting) filler formulation directly into a defect in a dermal layer, optionally filler formulations of different consistencies are used depending on the severity of the defect or the desired effect, optionally RHA2™ or equivalent filler is used for less severe defects and RHA4™ or equivalent is used for more pronounced defects; and/or
the method further comprises, for example, to recontour the eyelid-cheek junction, administering (or infiltrating, or injecting) filler formulation with least one retrotracing line in the area of the eyelid-cheek junction, and optionally the filler formulation comprises an RHA1™ or equivalent.

In alterative embodiments, provided are kits comprising a plurality of syringes having contained therein filler formulation comprising a sterile, crosslinked polysaccharide,
wherein the sterile, crosslinked polysaccharide has a crosslinker rate, or Degree of Modification (MoD), or rate of crosslinking, of between about 0.1% and 10%, 0.4% and 8%, 0.8% and 6%, 1% and 5%, or about 1.5% and 4%, or between about 2% and 4%, or the filler has a crosslinker rate (MoD) of about 0.1%, 0.2%, 0.3%, 0.4%, 0.5%, 0.6%, 0.7%, 0.8%, 0.9%, 1%, 1.5%, 2%, 2.5%, 3%, 3.5%, 4% or 4.5%,
wherein optionally each of the plurality of syringes has an adjustable needle depth stop to ensure that the filler formulation is substantially only injected into a superficial fat layer in the skin of the face,
and optionally each of the plurality of syringes comprises a filler formulation having a different crosslinking Degree of Modification (MoD).

In alterative embodiments of kits as provided herein:
the kit comprises at least one of the following syringes:
(a) a syringe comprising or having contained therein a filler formulation comprising an RHA1™ filler polysaccharide or equivalent filler polysaccharide having a Degree of Modification (MoD), or degree (amount) of crosslinking, of between about 0.1% and 2%, or between about 0.5% and 1.7%, or about 1.5%;
(b) a syringe comprising or having contained therein a filler formulation comprising an RHA2™ filler polysaccharide or equivalent filler polysaccharide having a Degree of Modification (MoD), or degree (amount) of crosslinking, of between about 2.5% and 3.2%, or between about 2.8% and 3.2%, or about 3.1%;
(c) a syringe comprising or having contained therein a filler formulation comprising an RHA3™ filler polysaccharide or equivalent filler polysaccharide having a Degree of Modification (MoD), or degree (amount) of crosslinking, of between about 3.3% and 3.8%, or between about 3.4% and 3.7%, or about 3.6%; and/or
(d) a syringe comprising or having contained therein a filler formulation comprising an RHA4™ filler polysaccharide or equivalent filler polysaccharide having a Degree of Modification (MoD), or degree (amount) of crosslinking, of between about 3.9% and 4.5%, or between about 4% and 4.3%, or about 4%,
wherein optionally the kit further comprising instructions for practicing a method for facial tissue engineering comprising administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face.

In alternative embodiments, provided are uses of kits as provided herein for methods of facial tissue engineering, wherein the methods comprise administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face.

In alternative embodiments, provided are kits as described herein for use in methods of facial tissue engineering, wherein the methods comprise administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 illustrates, in a schematic perspective view, the signs on the face due to aging facial tissues, as discussed in further detail, below.

FIG. 2 illustrates, in a schematic perspective view, the area defined as mid-face, as discussed in further detail, below.

FIG. 3 illustrates, in a schematic perspective view, the distinction between medial and lateral areas of the mid-face, as discussed in further detail, below.

FIG. 4 illustrates, in a schematic front elevation view, the structure of the skin, as discussed in further detail, below.

FIG. 5 illustrates, in a schematic perspective view, the distinction between the superficial fat compartments of part of the face, as discussed in further detail, below.

FIG. 6 illustrates, in a schematic perspective view, the distinction between some ligaments of part of the face, as discussed in further detail, below.

FIG. 7, FIG. 9, FIG. 10 illustrate, in a schematic perspective view, injection sites for some phases of exemplary methods as provided herein, as discussed in further detail, below.

FIG. 8 illustrates, in a schematic and enlarged manner, the correlation between injection sites for an exemplary embodiment, as discussed in further detail, below.

FIG. 11 illustrates an image of a patient and the site of injection for a exemplary phase of recontouring the eyelid-cheek junction, or phase 4, of an exemplary embodiment of a method as provided herein, as discussed in further detail, below.

FIG. 12 and FIG. 13 illustrate the effects on a patient, respectively before and after, of a treatment following an exemplary embodiment of a method as provided herein, as discussed in further detail, below.

FIG. 14 and FIG. 15 illustrate, the effects on a patient, respectively before and after, of a treatment following an exemplary embodiment of a method as provided herein, as discussed in further detail, below.

FIG. 16, FIG. 17, and FIG. 18 schematically illustrate three different embodiments of exemplary methods as provided herein, as discussed in further detail, below.

Like reference symbols in the various drawings indicate like elements.

### DETAILED DESCRIPTION

Provided are methods for modifying or engineering a facial profile, or any dermal defect (for example, on an arm or a leg), or for treating skin or facial volume defects. In alternative embodiments, provided are methods that comprise administration of cross-linked polysaccharide polymers, or polysaccharide polymer salts, such as hyaluronic acid-based formulations, wherein the cross-linked polysaccharide polymers, or polysaccharide polymer salts, or hyaluronic acid, have varying degrees of modification, or are cross-linked at low levels. In alternative embodiments, provided are methods for treating or ameliorating any dermal, for example, facial, volume defect and/or any unwanted static or dynamic lines (lines that are caused by facial expressions such as smiling, laughing, frowning or squinting), including crow's feet, forehead line or any unwanted facial line. In alternative embodiments, methods as provided herein administer cross-linked fillers (for example, cross-linked polysaccharide polymers, or polysaccharide polymer salts, such as hyaluronic acid-based formulations) that are injected superficially at predetermined sites on the skin, for example, injected at facial injection sites, such as into the mid-facial region, without reaching the deep fat layer. In alternative embodiments, methods as provided herein treat or ameliorate or modify unattractive nasolabial folds (NLF) (or indentation lines on either side of the mouth that extend from the edge of the nose to the mouth's outer corners, which become more prominent when people smile, and these folds can deepen with age).

In alternative embodiments, the cross-linked polysaccharide polymers used to practice methods as provided herein are “resilient”, or have a crosslinker rate (Degree of Modification (MoD)), or rate of crosslinking, of only between about 0.1% and 5%, 0.2% and 4.5%, 0.4% and 3%, 0.8% and 2%, 1% and 1.5%, or about 1.5% and 4%, or between about 2% and 3%.

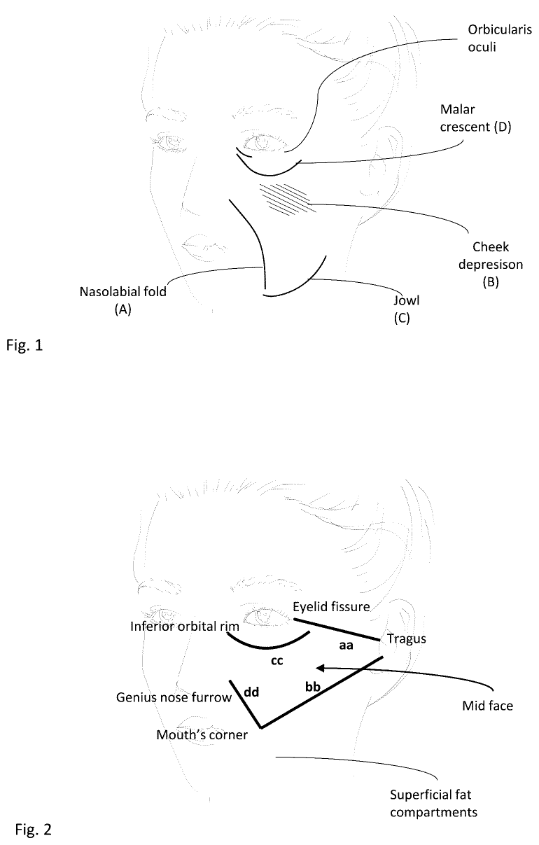

In alternative embodiments, provided are methods for treating or ameliorating facial volume defect and/or any unwanted facial static or dynamic lines by administering fillers in the mid-face area of the face, where the fillers are injected at or into the layer of superficial fat. In alternative embodiments, injection in this superficial fat layer represents an exclusive or main focus of treatment, and is particularly suitable for the treatment of facial volume defects in patients of younger age, for example, people aged between the second and third decade. FIG. 1 and FIG. 2 illustrate facial areas most subject to tissue aging (and on which the signs of time tend to show more of their effects at a superficial level) that can be treated using methods as provided herein, including a malar crescent (FIG. 1D), cheek depression (FIG. 1B), jowls (FIG. 1C) and/or nasolabial folds (FIG. 1A). For example, methods as provided herein are used to ameliorate the appearance of a nasolabial fold (FIG. 1A), which tends to become more pronounced, as well as the cheek muscles tend to relax leaving a depression (FIG. 1B) in the center of the cheek, resulting in a lowering of the cheek (FIG. 1C). In addition, age-related accentuating and thickening of a malar crescent (FIG. 1D) is treated or ameliorated using methods as provided herein.

In alternative embodiments, provided are methods for treating or ameliorating facial volume defect and/or any unwanted facial static or dynamic lines in the mid-face or mid-facial area, for example, as defined by Cotofana et al., Aesthetic Surgery Journal, 2021, or as illustrated in FIG. 2. In alternative embodiments, the mid-face or mid-facial area is delimited at the top by a line (aa) running from the upper margin of the tragus to the lateral canthus of the palpebral fissure, and at the bottom by a line (bb) running from the lower margin of the tragus to the corner of the mouth. The lateral limits of the mid-face are defined by the inferior orbital eyelid (cc) at the top, and the nasogenian furrow (dd) at the bottom.

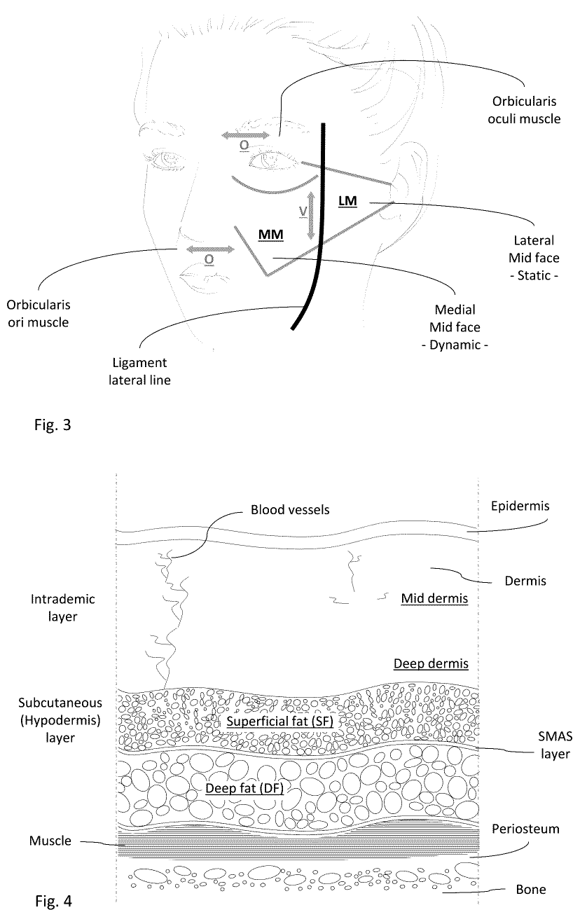

In alternative embodiments, for treatment considerations, the mid-face is divided into two areas: a medial area (MM), which is characterized by greater dynamism, and a lateral area (LM), also called static one, being characterized by reduced dynamism compared to the medial area. These two areas are separated by the lateral line of ligaments, as shown in FIG. 3.

In alternative embodiments, the medial part (MM) of the mid-face, which is characterized by the presence of mimic muscles responsible for the intense dynamic activity of the medial mid-face, is treated. It is precisely because of this different dynamic activity that the first signs of aging occur in the medial part of the mid-face, and in particular in the areas of transitions, i.e., in correspondence of the upper and lower boundaries of the mid-face. An overlap of muscle vectors occurs at these boundaries. Specifically, muscle vectors act mostly in a vertical direction in the mid-face, and indicated in FIG. 3 by the arrow V, whereas in the neighboring areas muscle vectors act mostly in a horizontal direction, indicated in FIG. 3 by the arrow O. Muscle vectors acting mostly in a horizontal direction are represented by the orbicularis oculis, and orbicularis oris muscle, respectively, at the upper and lower mid-face boundaries, as shown in FIG. 3.

In alternative embodiments, methods as provided herein treat or ameliorate furrows and wrinkles on the face and loss of facial volume caused by, for example, the overlapping of muscle vectors that act horizontally with those that act vertically, where with the advance of time and years determines a thickening of furrows and wrinkles on the face and the loss of facial volume. In particular, the dynamic activity of the medial mid-face contributes to the formation of the first and most relevant signs of facial aging, such as the progressive demarcation of the eyelid malar junction with the orbit-eyelid furrows, and the nasogenian demarcation. These signs of aging are dominant in the young female population, particularly in the second and third decades (28 to 40 years old). This results in an aging of the superficial fat (SF) compartments of the face—with a consequent atrophy even before that of the deep ones (DF), at the bone level in FIG. 4.

In alternative embodiments, methods as provided herein focus exclusively on the superficial fat (SF) layer of the face, and in particular in the transition areas of the mid-face, meaning by transition areas the eyelid-malar junction (cc) and/or the nasogenian demarcation (dd), which delimit the mid-face, as illustrated in FIG. 2. In alternative embodiments, methods as provided herein are particularly effective for treating or ameliorating facial signs of aging by focusing on the SF layer, and because signs of aging become more pronounced when the fat atrophies. At the superficial level, the fat, which is subject to a greater dynamic action, atrophies more easily and more quickly, one reason methods as provided herein primarily focus treatment administration at the superficial fat level.

In alternative embodiments, methods as provided herein focus treatment in transition areas, which are those with the greatest dynamic factor because it is at these transition areas that the opposing dynamics of vertical contraction of the mid-face and horizontal contraction of the two border areas, as delimited by the orbicularis muscles of the eye and the muscles of the mouth, meet.

In alternative embodiments, methods as provided herein make it possible to restore mobility and volume to the tissues in an effective way, i.e. with a reduced number of interventions and with a low risk of damage to the treated area. In alternative embodiments, methods as provided herein can reduce the signs of aging in the mid-face by acting primarily at the level of superficial fat (SF); this reduces the amount of filler that needs to be injected to achieve a desired result.

In addition, methods as provided herein make it possible to increase the efficiency of the filler itself because layers closer to the epidermis are targeted for treatment, thus achieving a change in facial volume in predetermined points with a lower amount of filler (as compared to injections in deeper layers using currently known methods).

In alternative embodiments, methods as provided herein focus treatment on the superficial plane, subcutaneous or intradermal; the treated “superficial” plane has, with the exception of the facial artery and its terminal branch at the side of the nose, small branches of blood vessels which are almost impossible to cannulate with needle or cannula, thus making the injection of filler safer using methods as provided herein. In fact, practicing methods as provided herein reduces the probability of hematoma in the days following the treatment, unlike injections made at deep level (the deep tissue of the skin is in fact the plane of the large arterial branches of the mid-face, which include the infra-orbital artery, the zygomatic-facial artery; therefore, injections into the center of the mid-facial district increase the likelihood of ischemic vascular events if performed deep, because this is the level of the larger caliber arterial branches that are more easily cannulated by cannula or needle).

In alternative embodiments, methods as provided herein use cross-linked filler injected or administered superficially, primarily into the superficial fat (SF) layer of the skin, into the periocular and/or into the mid-facial region, without reaching the deep fat layer.

In some embodiments, methods further or additionally comprise injecting a filler into the dermal layer of the skin.

In alternative embodiments, methods as provided herein comprise injecting a filler into a predefined injection site, for example:

In alternative embodiments, methods as provided herein comprise injecting a filler into a primary site: an exemplary injection site is designated the primary site, and is located at the zygomatic cutaneous ligament (ZCL), shown in FIG. 6. In one embodiment, the primary site is represented by the G point, as defined for example by Casabona et al. 15 Dec. 2019, J. Cosmetic Dermatology, and described below.

In alternative embodiments, methods as provided herein comprise injecting a filler into a lateral injection site: in one embodiment injection sites (up to three injection sites) are at the Zygomatic cutaneous ligament, designated lateral injection sites. In alternative embodiments, method comprise two additional injection sites in addition to the primary site, which can be located: one upstream and one downstream of the primary site.

In alternative embodiments: at least one injection site, other than the primary injection site, is identified at the periocular region. In alternative embodiments, fillers are administered at three (3) injection sites, defined at the periocular region, designated as central injection sites.

In alternative embodiments, fillers that are injected comprise a “resilient” cross-linked polysaccharide polymer, or polysaccharide polymer salt, for example, a hyaluronic acid-based filler: the filler “resilient” based on its comprising a cross-linked polysaccharide polymer (for example, hyaluronic acid) which is adapted to resist to the stress of movement for a longer period of time. In alternative embodiments, a “resilient” filler as provided and as used in methods as provided herein has a crosslinker rate (Degree of Modification (MoD)), or rate of crosslinking, of between about 0.1% and 4.5%, 0.2% and 4%, 0.4% and 3%, 0.8% and 2%, 1% and 1.5%, or about 1.5% and 4%, or between about 2% and 3%, or the filler has a crosslinker rate of about 0.1%, 0.2%, 0.3%, 0.4%, 0.5%, 0.6%, 0.7%, 0.8%, 0.9%, 1%, 1.5%, 2%, 2.5%, 3%, 3.5%, 4% or 4.5%.

In alternative embodiments, fillers that are injected comprise a cross-linked polysaccharide polymer, or polysaccharide polymer salt, for example, hyaluronic acid, concentration of between about 10 mg/mL and 35 mg/mL, or between about 15 mg/mL and 30 mg/mL, or between about 20 mg/mL and 25 mg/mL.

In alternative embodiments, fillers used in methods as provided herein comprise cross-linked polysaccharide polymers, or polysaccharide polymer salts, including for example any physiologically acceptable salt such as the sodium salt, potassium salt, zinc salt and/or silver salt, and mixtures thereof.

In alternative embodiments, a crosslinking agent for making cross-linked polysaccharide polymers comprises (or are selected from the group consisting of): epoxides, aldehydes, polyaziridyl compounds, divinyl sulphone (DVS) and/or butanediol diglycidyl ether (BDDE), noting that butanediol diglycidyl ether has the lowest toxicity among the standard crosslinking agents.

In alternative embodiments, a crosslinking agent for making cross-linked polysaccharide polymers comprises an epoxy crosslinking agent, which can be bifunctional or multifunctional, for example, can comprise: 1,4-butanediol diglycidyl ether (BDDE), diepoxyoctane or 1,2-bis(2,3-epoxypropyl)-2,3-ethylene, 1,4-bis (-epoxypropoxy) butane, 1,4-bisglycidyloxybutane, 1,2-bis (2,3-epoxypropoxy) ethylene, and/or 1-(2,3-epoxypropyl)-2,3-epoxycyclohexane, and mixtures thereof.

In alternative embodiments, fillers used in methods as provided herein comprise, for example: RHA4™, RHA3™, RHA2™, by Revance (Nashville, TN), or equivalents, and/or RHA4™, RHA3™, RHA2™ by Teoxane, or equivalents. RHA1™, RHA2™, RHA3TM, RHA4™, or equivalents, which are dermal fillers naturally derived and absorbable. They are based on Hyaluronic Acid (HA), including for example sodium Hyaluronic Acid (NaHA), derived from a bacterial formation (for example, from Streptococcus equi bacterial strain), often cross-linked with 1,4-butanediol diglycidyl ether (BDDE). These formulations can be viscoelastic, sterile, non-pyrogenic, clear, colorless, homogeneous and biodegradable.

HA, or NaHA, as used in methods as provided herein is resilient because it has high molecular weight HA chains with low crosslinking (wherein the rate of crosslinking is only between about 0.1% and 4%, 0.4% and 3%, 0.8% and 2%, 1% and 1.5%, or about 1.5% and 4%, or between about 2% and 3%) to minimize the degradation of HA chains during the manufacturing process and reduce the Degree of Modification (MoD), or degree (amount) of crosslinking, of polysaccharide, for example, HA (or NaHA), in the final product.

The MoD helps to improve the adaptability to skin movements. The resulting high molecular weight chains forms a network of entangled polysaccharide (for example, HA) fibers which require fewer 1,4-butanediol diglycidyl ether (BDDE)-covalent bonds for stabilization. As a result, the MoD of polysaccharide (for example, HA, or RHA™) fillers remain low (2% to 4%) compared to most monophasic gels (5% to 10%), allowing the less rigidly crosslinked polysaccharide (or HA) chains to interact and slide dynamically while maintaining in vivo durability.

| | HA (mg/ml) | MoD (%) | BDDE | Note |
|---|---|---|---|---|
| RHA1: | 15 | about 1.5 | + | - crosslinks, less rigid HA chain, long chains |
| RHA2: | 23 | about 3.1 | ++ | - crosslinks, less rigid HA chain, long chains |
| RHA3: | 23 | about 3.6 | +++ | - crosslinks, less rigid HA chain, long chains |
| RHA4: | 23 | about 4 | ++++ | - crosslinks, less rigid HA chain, long chains |
| Non-resilient: | >15 | 5-10 | +++++++ | + crosslinks, more rigid HA chain, fragmented chains |

In alternative embodiments, an RHA1™ filler formulation or equivalent filler formulation comprises a cross-linked polysaccharide (for example, cross-linked with BDDE) having a Degree of Modification (MoD), or degree (amount) of crosslinking, of between about 0.1% and 2%, or between about 0.5% and 1.7%, or about 1.5.

In alternative embodiments, an RHA2™ filer formulation or equivalent filler formulation comprises a cross-linked polysaccharide (for example, cross-linked with BDDE) having a Degree of Modification (MoD), or degree (amount) of crosslinking, of between about 2.5% and 3.2%, or between about 2.8% and 3.2%, or about 3.1%.

In alternative embodiments, an RHA3™ filler formulation or equivalent filler formulation comprises a cross-linked polysaccharide (for example, cross-linked with BDDE) having a Degree of Modification (MoD), or degree (amount) of crosslinking, of between about 3.3% and 3.8%, or between about 3.4% and 3.7%, or about 3.6%.

In alternative embodiments, an RHA4™ filler formulation or equivalent filler formulation comprises a cross-linked polysaccharide (for example, cross-linked with BDDE) having a Degree of Modification (MoD), or degree (amount) of crosslinking, of between about 3.9% and 4.5%, or between about 4% and 4.3%, or about 4%.

In alternative embodiments, methods and kits as provided herein make, formulate and use cross-linked polysaccharides (for example, cross-linked with BDDE, optionally using HA), including using syringes and techniques, as described in for example: US pat app pub nos. 20210259943 and 20210308055, and U.S. Pat. Nos. 11,260,015 and 11,324,672.

In alternative embodiments, methods as provided herein comprise:

### Treatment of the Nasolabial Fold, Volumizing the Cheek

(a) a first phase (step or phase 1), called lift & support, comprising a first sub-phase (1a) of lifting and a second sub-phase (1b) of support, in which the lower orbital eyelid and the upper part of the cheek are treated. This step results in the stretching and support of the mid-facial tissues, allowing indirect treatment of the nasolabial fold, and bringing a volumizing effect to the cheek.

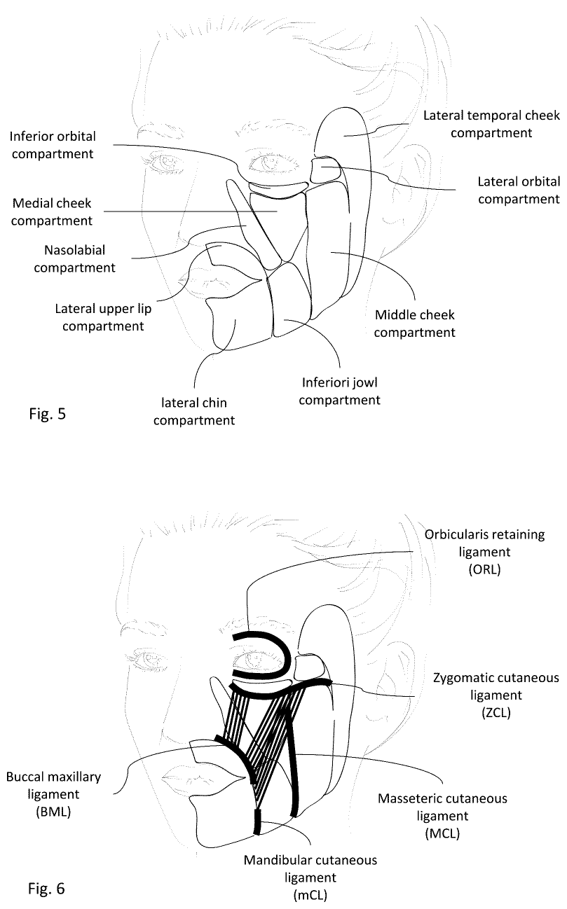

In the lift phase (1a), at least one bolus (for example, from 0.05 mL (0.05 cc) filler to 0.3 mL (0.3 cc) filler) is injected at the lateral facial area, and more specifically, into the middle cheek fat compartment near (or at a site between about 0.5 to 5 mm, or within about 1, 2, 3, 4 or 5 mm) the zygomatic cutaneous ligament (ZCL), shown in FIG. 5. In alternative embodiments, two or three boluses are administered, at the upper zone of the middle cheek fat compartment near (or at a site between about 0.5 to 5 mm, or within about 1, 2, 3, 4 or 5 mm) the zygomatic cutaneous ligament (ZCL).

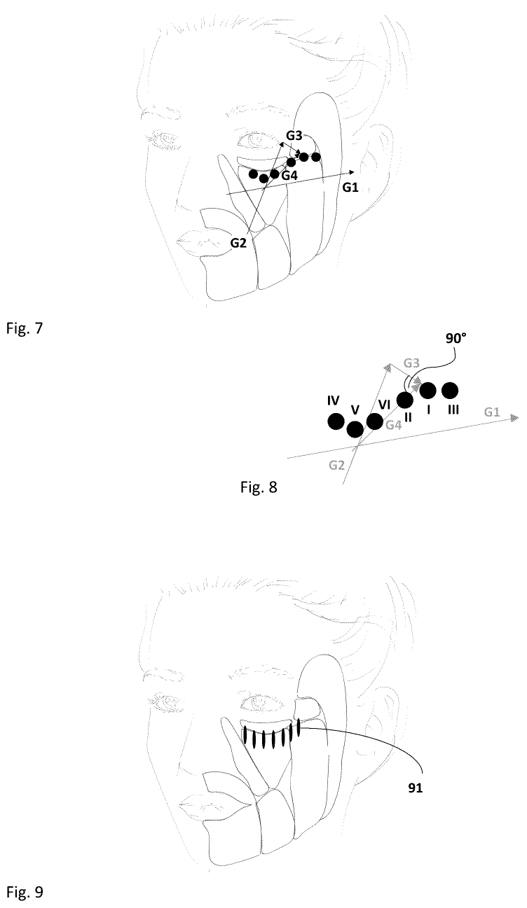

In alternative embodiments, one, two or three or more boluses of about 0.1 mL (0.1 cc) filler, or between about 0.5 and 1.5 ml, are injected subcutaneously into the superficial fat, referred to as lateral boluses I, II, III, in FIG. 7, as illustrated in FIG. 7. In some embodiments, not illustrated, the three boluses comprise different volumes ranging from 0.05 mL (0.05 cc) filler to 0.3 mL (0.3 cc) filler.

In alternative embodiments, the two or three lateral boluses are equidistant from each other. In alternative embodiments, the first bolus (I) is injected at the Gpoint, as identified and represented in FIG. 7 and FIG. 8.

In alternative embodiments, the Gpoint is derived by plotting:
a first connecting line (G1) between the lower end of the nasal wing and the tragus,
a second connecting line (G2) between the lateral end of the eye and the corner of the mouth,
a third connecting line (G3) that crosses the second connecting line G2, and
a fourth connecting line (G4) drawn from the intersection of the first G1 and the second G2 connecting line until it intersects the third G3 connecting line at a 90° angle.

The point of intersection of the line G3 with the line G4, at the point where they form an angle of 90°, defines the so-called Gpoint. The Gpoint has the characteristic of maximizing the lifting effect with the same filler used, meaning by ‘lifting effect’ the lifting and distension of facial tissues.

At this stage, at least one (1) bolus is injected at the Gpoint, at the level of the superficial fat in the middle cheek fat compartment.

In alternative embodiments, a second bolus is injected into the middle cheek compartment, anteriorly and lower than the Gpoint, following the zygomatic cutaneous ligament (ZCL), as shown in FIG. 8.

In alternative embodiments, a third bolus is injected into the middle cheek compartment, posterior to the Gpoint, following the zygomatic cutaneous ligament (ZCL), as shown in FIG. 8.

The purpose of lateral boluses is to induce a lifting effect of the orbital ligament (ORL) and zygomatic-cutaneous ligament (ZCL), shown in FIG. 6. The injection of the filler in the three indicated sites—three (3) lateral boluses I, II, III—also allows to obtain an indirect improvement in the nasolabial area with the reduction of the nasolabial wrinkle.

In alternative embodiments, the lateral boluses are injected using a 25 gauge (G) cannula or 27 G needle.

In alternative embodiments, to inject with a needle in the subcutaneous layer, the skin is penetrated tangentially to the skin, just before the predetermined injection site of a few millimeters. For this purpose, in alternative embodiments, the cannula or needle is inserted into the skin at an angle of 5 to 50 degrees, or at 10 to 15 degrees. In either case, for example, with the filler syringe provided with needle or cannula, the user can check the depth of the plane in which he/she is operating by lifting the needle/cannula. To check the right plane, he/she lifts the needle right before injecting and verify the thickness of the tissue overlying it.

The fact of operating superficially allows to avoid the intra-arterial perforation of the zygomatic-facial artery, which emerges inferiorly and medially in the malar area, at a deep level of the periosteum.

In the support phase (1b), at least one bolus is injected at the medial facial compartment of the cheek in the area of greatest atrophy, identified as the greatest depression in the periocular inferior eyelid area. In alternative embodiments, three (3) boluses are used following the zygomatic cutaneous ligament (ZCL), into the medial cheek compartment.

In alternative embodiments, three (3) boluses of about 0.1 mL (0.1 cc) are injected subcutaneously into the superficial fat of the medial cheek compartment, referred to as central boluses IV, V, VI. In alternative embodiments, the 3 central boluses are arranged at the three vertices of a triangle to form a kind of V in the superficial fat of the medial cheek compartment in the area of major atrophy of the muscles and fat. In alternative embodiments, the 3 central boluses are equidistant from each other. In alternative embodiments, the three central boluses are injected in any order. In alternative embodiments, the three central boluses allow for cheek support, and thus avoid cheek depression. In alternative embodiments, the three central boluses allow for improved distension and reduced folding of the upper nasolabial fold.

In alternative embodiments, injection at the level of the medial superficial compartment of the cheek allows to reduce, and at the limit avoid, the risk of perforating the infra-orbital artery, which emerges from the maxillary bone in the injection areas, since the artery keeps in contact with the periosteum, that is at a deeper layer.

In alternative embodiments, a high GPrime filler is used in phase 1. Suitable fillers for the phase 1 are, for example, the RHA4™ or equivalent filler by Teoxane Laboratories (Geneva, Switzerland) or Revance (Nashville, TN) (i.e. high GPrime). Other suitable, exemplary fillers are for example: RHA3™ or equivalent filler by Teoxane or Revance (i.e. medium GPrime), and RHA2™ or equivalent filler by Teoxane or Revance (i.e. low GPrime).

### Cheek Contouring

In order to have a further improved effect, in alternative further embodiments, methods as provided herein comprise additional step of cheek contouring, designated phase 2, as illustrated in FIG. 9. At this stage, along the upper part of the cheek, optionally between the inferior orbital compartment and the middle cheek compartment, a filler is infiltrated (administered) following at least one retrotracing line (see 91, FIG. 9) arranged as a bridge between the two mentioned superficial fat compartments (bridging technique of the defect).

In alternative embodiments, about three (3) to ten (10) retrotracing lines, or between about 6 to 8 retrotracing lines, are practiced, in which the filler is injected or administered. In alternative embodiments, the filler used for the retrotracing lines is the same as that used in the phase 1. For example, the filler used in phase 2 comprises RHA4™ or equivalent.

In further embodiments, the filler used for the retrotracing lines is different than that used in step 1.

In alternative embodiments, to enhance the plumping effect of the lower eye contour, a filler is injected according to retrotracing lines interposed at the phase 1 injection sites, i.e., in areas adjacent to the boluses injected in phase 1. This optimizes the transition between the central (sites IV-VI) and lateral (sites I-III) boluses of phase 1. This can improve the treatment of areas of cheek atrophy, and the indirect distension effect in the nasolabial area.

### Nasolabial Treatment

In further optional embodiments, a method as provided herein comprises a nasolabial treatment, designated phase 3, as illustrated as an example in FIG. 10. At this stage, the filler is infiltrated into the deep or mid dermis (see FIG. 4) at the nasolabial fold, that is on a more superficial layer, compared to the injections described in phase 1 and 2, above. In alternative embodiments, the filler is infiltrated (administered, injected) with at least one retrotracing line (see 101, FIG. 10).

In alternative embodiments, infiltration is performed using a bridging technique of the defect. In other words, the needle, or cannula, enters laterally into the cheek side of the defect, passes through it, and into the lip side. Here the release of the filler is initiated in a linear retreating fashion, also called retrotracing mode. The nasolabial fold is thus crossed transversely by the filler, and the release is finished as soon as it reaches the cheek side.

To improve the volumizing effect, in some embodiments, is provided one or more boluses (see 102, FIG. 10), injected at deep or mid intradermal level (deep/mid-dermis). In alternative embodiments, one, two, three or more boluses (102, FIG. 10) are of 0.05 mL in size.

In alternative embodiments, in phase 3, an RHA3™ or equivalent filler injected into a deep or mid dermis plane is used. Optionally, a 27G needle or 25G cannula is used.

In alternative embodiments, phase 3, about two (2) to seven (7) retrotracing lines, optionally about three (3) retrotracing lines, are performed to achieve appreciable, or desired, defect correction.

In some embodiments, methods as provided herein comprise some finishing touches (see 103, FIG. 10), in which the filler is injected (or administered) directly into the defect in the dermal layer, for example, to fill in a wrinkle. In such situations, optionally, fillers of different consistencies are used depending on the severity of the defect or the desired effect. For example, RHA2™ or equivalent filler can be used for less severe defects and RHA4™ or equivalent for more pronounced defects.

In alternative embodiments, in the terminal part of the defect, filler is injected (or administered) directly into the wrinkle in the most superficial plane possible, such as the mid-dermis.

In alternative embodiments, the nasolabial treatment phase (phase 3) replaces the cheek contouring phase (phase 2).

In other embodiments, the method, in addition to the lift and support phase (phase 1), includes the cheek contouring phase (2) and subsequently or previously the nasolabial treatment treatment phase (3).

In alternative embodiments, methods as provided herein comprise three (3) steps performed in the following order:
a 1st phase of lift (1a) and support (1b) in which the lower orbital eyelid and the upper part of the cheek are treated, in order to stretch and support the tissues, allowing to treat indirectly the nasolabial fold;
a 2nd phase of cheek contouring in which the cheek area is treated, to increase the volume and support effect of the tissues in the upper part of the cheek, helping to accentuate the distension of the nasolabial fold; and
3rd phase of nasolabial treatment in which the area of the nasolabial fold is treated, acting directly at the level of the defect.

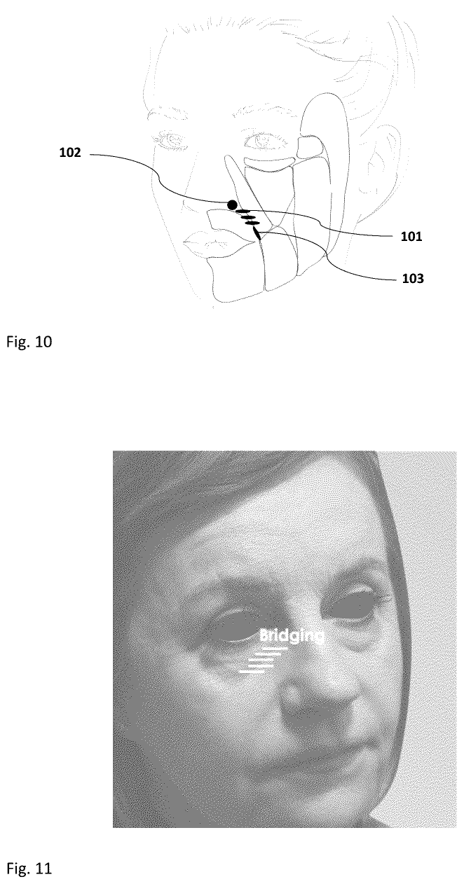

In further embodiments, methods as provided herein, in addition to the phase of lift and support (phase 1), further comprises the phase of recontouring the eyelid-cheek junction (phase 4), as shown for example in FIG. 11. This phase comprises at least one retrotracing line in the area of the eyelid-cheek junction to recontouring it. In phase 4, a filler with low cohesivity is used. This filler is one dedicated to the treatment of the infra-orbital area, such as RHA1™ by Teoxane and/or Revance, or equivalents. In alternative embodiments, filler is injected, entering from the Gpoint, with a cannula.

In alternative embodiments, in this phase micro-threads of roughly 0.02 ml to 0.03 ml length are delivered under the orbicularis muscle for the passage across the infra-orbital hollow, and crossing the local ligaments, such as the tear trough and orbital retaining ligament. In alternative embodiments, between about 2 to 10, or 3 to 8, or 4 to 6, passages are made across the area to support the ligaments and recontour the eyelid-cheek junction. In alternative embodiments, the technique used here is characterized as ‘bridging’ of the tear trough because it consists in bridges of filler that support the muscle delivered across the valley (the tear trough deformity). The advantage of using this technique is that it allows use of less product in a difficult area at high risk for complications; the average quantities of filler in the tear trough are in average between 1 and 2 ml at most, compared to the 3 to 5 ml of the technique of injection directly within the defect.

In alternative embodiments, methods as provided herein also comprise an oral commissures and marionettes fill-in phase for treating the small lines around the mouth.

In alternative embodiments, methods as provided herein are applied to both female and male patients.

### Products of Manufacture and Kits

Provided are products of manufacture and kits for practicing methods as provided herein, including for example syringes comprising filler as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more, for example, of a referenced amount of a composition, or when describing a method for facial tissue engineering comprising administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face, the claim encompassed embodiments where at least about 75%, 80%, 85%, 90%, 91%, 92%, 93%, 94%, 95%, 96%, 97%, 98%, 99% or 99.5%, or more of the crosslinked polysaccharide is injected into a superficial fat layer in the skin of the face.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Exemplary Protocols—Treating the Nasolabial Fold, Inferior Periocular Depression and Cheek

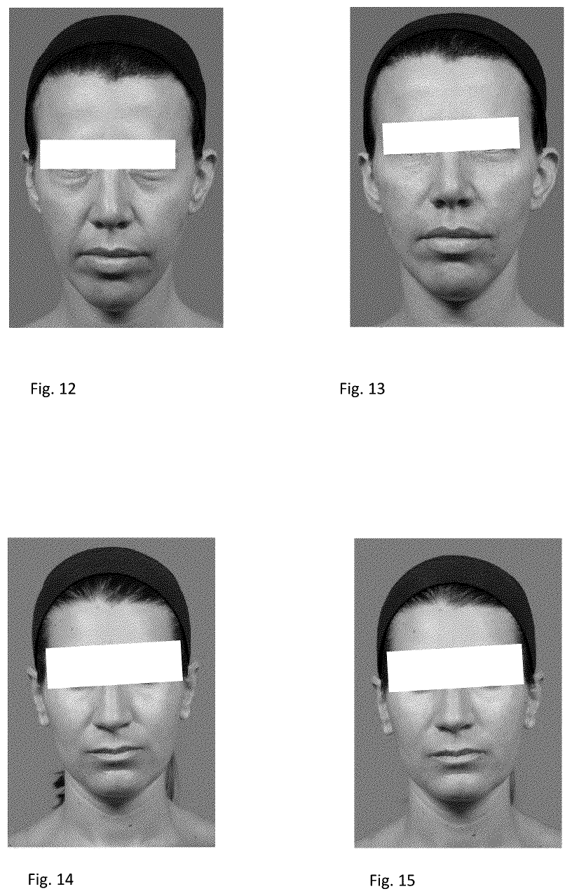

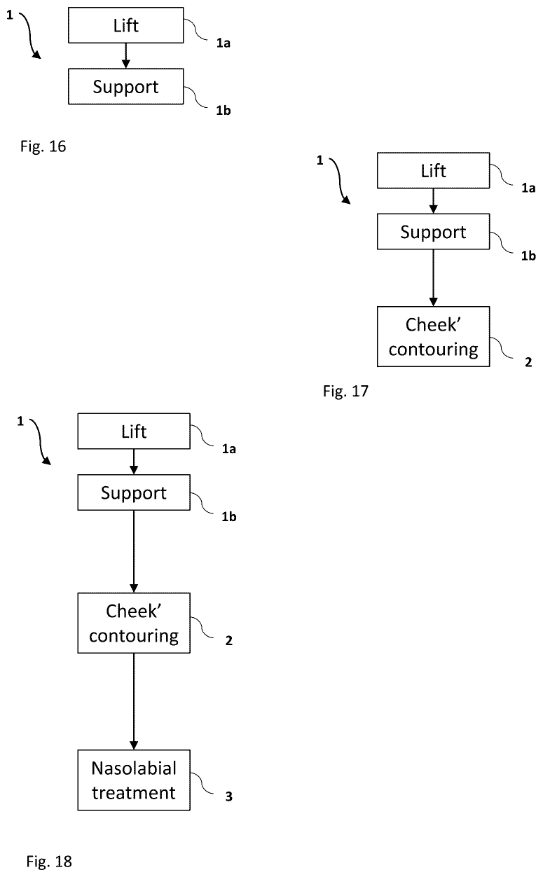

This example demonstrates an exemplary protocol as provided herein, where a female 33 year-old patient was treated using a method as provided herein, as illustrated in FIG. 12 and FIG. 13.

The treatment involved the injection of resilient fillers (as defined above) in the total quantities indicated in the column of Table 1, below, labeled ‘volume’ (also, nasolabial folds labeled NLF), with the product indicated in the column labeled ‘Product’, for the respective phases and treatment areas indicated in the remaining columns.

Phase 1 was repeated to add some filler after some days.

As can be seen in the figures, before and after treatment, respectively FIG. 12 and FIG. 13, the patient shows a visible reduction of the nasolabial fold and the inferior periocular depression, with also a volumization of the cheek.

TABLE 1

| Product | Volume | Treatment Area | Phase |
|---|---|---|---|
| RHA 4 | 1.0 mL | Indirect NLF | 1 |
| RHA 2 | 1.0 mL | Indirect NLF | 2 |
| RHA 3 | 1.0 mL | Direct NLF | 3 |
| RHA 2 | 0.6 mL | Oral Commissures & | — |
| | | Marionettes | |

### Example 2: Exemplary Protocols—Treating the Nasolabial Fold, Increasing Cheek Volume

This example demonstrates an exemplary protocol as provided herein, where a female 28 year-old patient was treated using a method as provided herein, as illustrated in FIG. 14 and FIG. 15.

As described in Example 1, the treatment involved the injection of resilient fillers (or having a crosslinking rate of between about 0.1% and 4%, 0.4% and 3%, 0.8% and 2%, 1% and 1.5%, or about 1.5% and 4%, or between about 2% and 3%) in the quantities, products, areas and method phases as indicated in the table below (Table 2) (also, nasolabial folds labeled NLF). Phase 1 was repeated to add some filler after some days.

As illustrated in FIG. 14 and FIG. 15, before and after the treatment, see respectively FIG. 14 and FIG. 15, the patient shows an accentuated reduction of the nasolabial fold and an improvement of the cheek volume.

TABLE 2

| Product | Volume | Treatment Area | Phase |
|---|---|---|---|
| RHA 4 | 1.0 mL | Indirect NLF | 1 |
| RHA 2 | 1.0 mL | Indirect NLF | 2 |
| RHA 3 | 0.8 mL | NLF | 3 |

A number of embodiments of the invention have been described. Nevertheless, it can be understood that various modifications may be made without departing from the spirit and scope of the invention. Accordingly, other embodiments are within the scope of the following claims.

What is claimed is:

1. A method for facial tissue engineering comprising administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face.

2. The method of claim 1, wherein the crosslinked polysaccharide comprises a crosslinked hyaluronic acid polysaccharide.

3. The method of claim 1, wherein the crosslinked polysaccharide comprises a salt of a polysaccharide, and optionally the crosslinked polysaccharide salt comprises a sodium salt, a potassium salt, a zinc salt and/or a silver salt or a mixture thereof.

4. The method of claim 1, wherein the crosslinked polysaccharide has a crosslinker rate, or Degree of Modification (MoD), or rate of crosslinking, of between about 0.1% and 10%, 0.4% and 8%, 0.8% and 6%, 1% and 5%, or about 1.5% and 4%, or between about 2% and 4%, or the filler has a crosslinker rate of about 0.1%, 0.2%, 0.3%, 0.4%, 0.5%, 0.6%, 0.7%, 0.8%, 0.9%, 1%, 1.5%, 2%, 2.5%, 3%, 3.5%, 4% or 4.5%.

5. The method of claim 1, wherein the polysaccharide is crosslinked with or using an epoxide, an aldehydes, a polyaziridyl compound, divinyl sulphone (DVS) and/or butanediol diglycidyl ether (BDDE), or a mixture thereof, and optionally the polysaccharide is crosslinked with or using: 1,4-butanediol diglycidyl ether (BDDE), diepoxyoctane or 1,2-bis(2,3-epoxypropyl)-2,3-ethylene, 1,4-bis(-epoxypropoxy) butane, 1,4-bisglycidyloxybutane, 1,2-bis (2,3-epoxypropoxy) ethylene, and/or 1-(2,3-epoxypropyl)-2,3-epoxycyclohexane, or a mixture thereof.

6. The method of claim 1, comprising administering or injecting subcutaneously into the superficial fat of the middle cheek compartment and/or medial cheek compartment (as illustrated in FIG. 5) an amount of filler formulation sufficient to cause stretching and supporting of the mid-facial tissues, thus allowing indirect treatment of the nasolabial fold (NLF), and bringing a volumizing effect to the cheek, and/or optionally, the method comprising:
(a) administering or injecting subcutaneously at least one bolus (optionally from about 0.05 mL (0.05 cc) to about 0.3 mL (0.3 cc) filler formulation) in or at a lateral facial area, and/or into a middle cheek fat compartment near, or at a site between about 0.5 to 5 mm, or within about 1, 2, 3, 4 or 5 mm of, the zygomatic cutaneous ligament (ZCL), optionally as shown in FIG. 5; and/or
(b) administering or injecting subcutaneously at least one bolus (optionally from about 0.05 mL (0.05 cc) to about 0.3 mL (0.3 cc) filler formulation) into the upper zone of the medial cheek fat compartment in the area of its major atrophy or depression.

7. The method of claim 1, comprising administering or injecting subcutaneously into the superficial fat in an upper zone of a middle cheek fat compartment at a site between about 0.5 to 5 mm, or within about 1, 2, 3, 4 or 5 mm, of a zygomatic cutaneous ligament (ZCL) a first bolus of the filler formulation comprising a volume from 0.05 mL (0.05 cc) filler to 0.3 mL (0.3 cc) of the filler formulation.

8. The method of claim 7, further comprising injecting filler formulation subcutaneously into the superficial fat one, two or three or more additional boluses (or injecting a second, third or fourth or additional bolus or boluses) in the upper zone of the middle cheek fat compartment, wherein optionally the one, two, three, four or more boluses of filler formulation are administered or injected in a volume of between about 0.05 ml to 1.5 ml.

9. The method of claim 8, wherein the second and/or third bolus or additional boluses of filler formulation is/are administered or injected subcutaneously into the superficial fat at sites as illustrated in FIG. 7 and FIG. 8 and/or approximately equidistant from previous administrations or injection of filler formulation.

10. The method of claim 7, wherein the first bolus of filler formulation is administered or injected at approximately at a Gpoint as derived by plotting:
a first connecting line (G1) between the lower end of the nasal wing and the tragus,
a second connecting line (G2) between the lateral end of the eye and the corner of the mouth,
a third connecting line (G3) that crosses the second connecting line G2, and
a fourth connecting line (G4) drawn from the intersection of the first G1 and the second G2 connecting line until it intersects the third G3 connecting line at a 90° angle.

11. The method of claim 10, wherein a second bolus of filler formulation is administered or injected into a middle cheek compartment, anteriorly and lower than the Gpoint, following the zygomatic cutaneous ligament (ZCL), as shown in FIG. 8, and/or a third bolus of filler formulation is administered or injected into the middle cheek compartment, posterior to the Gpoint, following the zygomatic cutaneous ligament (ZCL), as shown in FIG. 8.

12. The method of claim 1, further comprising injecting filler formulation subcutaneously into the superficial fat one, two or three or more additional boluses (or injecting a second, third or fourth or additional bolus or boluses) in the upper zone of the medial cheek fat compartment,
wherein optionally the one, two, three, four or more boluses of filler formulation are administered or injected in a volume of between about 0.05 ml to 1.5 ml.

13. The method of claim 1 further comprising administering (or injecting) in the upper part of the cheek, optionally between the inferior orbital compartment and the medial or middle cheek compartment, a filler formulation following at least one retrotracing line arranged as a bridge between the two superficial fat compartments,
and/or optionally the retrotracing lines are practiced in about three (3) to ten (10) retrotracing line sites, or between about 6 to 8 retrotracing line sites, wherein optionally the filler comprises RHA1™ or equivalent, RHA2™ or equivalent, RHA3™ or equivalent or RHA4™ or equivalent.

14. The method of claim 13 comprising administering (or injecting) filler formulation to enhance a plumping effect of a lower eye contour, wherein the filler formulation is injected according to retrotracing lines arranged between two adjacent boluses.

15. The method of claim 1 further comprising administering (or injecting) filler formulation into a deep or mid dermis at a nasolabial fold, and optionally the filler formulation is infiltrated (administered, injected) with at least one retrotracing line.

16. The method of claim 15 wherein the administering (or infiltrating, or injecting) filler formulation is performed using a bridging technique of the defect, and optionally a needle, or cannula, enters laterally into the cheek side of a defect, passes through it, and into the lip side, and release of the filler formulation is initiated in a linear retreating fashion, also called retrotracing mode, and the nasolabial fold is thus crossed transversely by the filler, and the release is finished as soon as it reaches the cheek side.

17. The method of claim 15, further comprising, to improve a volumizing effect, administering (or infiltrating, or injecting) one or more boluses filler formulation at a deep or mid intradermal level or deep/mid-dermis, optionally administering (or infiltrating, or injecting) one, two, three or more boluses, optionally 0.05 mL in volume size, and optionally the filler formulation comprises an RHA3™ or equivalent filler injected into a deep or mid dermis plane.

18. The method of claim 15, further comprising, to fill in a wrinkle, administering (or infiltrating, or injecting) filler formulation directly into a defect in a dermal layer, optionally filler formulations of different consistencies are used depending on the severity of the defect or the desired effect, optionally RHA2™ or equivalent filler is used for less severe defects and RHA4™ or equivalent is used for more pronounced defects.

19. The method of claim 1, further comprising, to recontour the eyelid-cheek junction, administering (or infiltrating, or injecting) filler formulation with least one retrotracing line in the area of the eyelid-cheek junction, and optionally the filler formulation comprises an RHA1™ or equivalent.

20. A kit comprising a plurality of syringes having contained therein filler formulation comprising a sterile, crosslinked polysaccharide,
wherein the sterile, crosslinked polysaccharide has a crosslinker rate, or Degree of Modification (MoD), or rate of crosslinking, of between about 0.1% and 10%, 0.4% and 8%, 0.8% and 6%, 1% and 5%, or about 1.5% and 4%, or between about 2% and 4%, or the filler has a crosslinker rate (MoD) of about 0.1%, 0.2%, 0.3%, 0.4%, 0.5%, 0.6%, 0.7%, 0.8%, 0.9%, 1%, 1.5%, 2%, 2.5%, 3%, 3.5%, 4% or 4.5%,
wherein optionally each of the plurality of syringes has an adjustable needle depth stop to ensure that the filler formulation is substantially only injected into a superficial fat layer in the skin of the face,
and optionally each of the plurality of syringes comprises a filler formulation having a different crosslinking Degree of Modification (MoD).

21. The kit of claim 20, wherein the kit comprises at least one of the following syringes:
(a) a syringe comprising or having contained therein a filler formulation comprising an RHA1™ filler polysaccharide or equivalent filler polysaccharide having a Degree of Modification (MoD), or degree (amount) of crosslinking, of between about 0.1% and 2%, or between about 0.5% and 1.7%, or about 1.5%;
(b) a syringe comprising or having contained therein a filler formulation comprising an RHA2™ filler polysaccharide or equivalent filler polysaccharide having a Degree of Modification (MoD), or degree (amount) of crosslinking, of between about 2.5% and 3.2%, or between about 2.8% and 3.2%, or about 3.1%;
(c) a syringe comprising or having contained therein a filler formulation comprising an RHA3™ filler polysaccharide or equivalent filler polysaccharide having a Degree of Modification (MoD), or degree (amount) of crosslinking, of between about 3.3% and 3.8%, or between about 3.4% and 3.7%, or about 3.6%; and/or
(d) a syringe comprising or having contained therein a filler formulation comprising an RHA4™ filler polysaccharide or equivalent filler polysaccharide having a Degree of Modification (MoD), or degree (amount) of crosslinking, of between about 3.9% and 4.5%, or between about 4% and 4.3%, or about 4%,
wherein optionally the kit further comprising instructions for practicing a method for facial tissue engineering comprising administering to an individual in need thereof a filler formulation comprising a sterile, crosslinked polysaccharide, wherein the crosslinked polysaccharide is administered by injection, and the crosslinked polysaccharide is substantially only injected into a superficial fat layer in the skin of the face.
